(19) United States
(12) Patent Application Publication
(10) Pub. No.: US 2025/0010062 A1
(43) Pub. Date: Jan. 9, 2025

(54) BRAIN STIMULATOR APPARATUS

(71) Applicant: Francisco Loyola, Perth (AU)

(72) Inventor: Francisco Loyola, Perth (AU)

(21) Appl. No.: 18/708,374

(22) PCT Filed: Nov. 10, 2022
(86) PCT No.: PCT/AU2022/051341

§ 371 Date: May 8, 2024

(30) Foreign Application Priority Data
Nov. 10, 2021 (AU) 2021903598

(51) Int. Cl. A61N 1/04; A61N 1/05; A61N 1/08; A61N 1/20; A61N 2/00

(52) U.S. Cl. CPC A61N 1/0484; A61N 1/0456; A61N 1/0502; A61N 1/0529; A61N 1/08; A61N 1/205; A61N 2/002; A61N 2/006

(57) ABSTRACT

A brain stimulator apparatus is described. The brain stimulator apparatus includes a power source; a control system; a plurality of electrodes; a wearable support for the electrodes; a number of electrical conductors electrically connecting the electrodes to the control system and/or to the power source. The control system is configured to control application of a voltage to the electrodes via the electrical conductors. The voltage applied stimulates one or more neurons in the wearer's brain.

### FIELD OF THE INVENTION

The present invention relates to a brain stimulator apparatus, such as to promote a preferred emotion (such as through release of neurotransmitters), manage or affect one or more neurological conditions and/or to target and stimulate/modify brain activity.

The present invention can be provided including a wearable brain stimulator, such as incorporated with headgear e.g. a hat or cap.

### BACKGROUND TO THE INVENTION

It is desirable to manage conditions related to the brain and brain function.

Conditions such as depression, anxiety, ADHD and other known neurobiological illnesses and neurodevelopmental disorders along with potentially dementia, currently are treated using medication and/or a combination of electric or magnetic therapy treatments. Using medication can enable a change in the release of neurotransmitters in the brain to produce a desired response in the individual being treated, because of mental illness or condition. Medication often has undesirable side-effects that can negate the positive effects of the medication, depending on the response of the individual. Often, the first medication given to a patient does not help the patient, and several different medications may be given or doses varied over many months until the patient is successfully well enough to function ‘normally’ again.

This type of medication treatment would be classified as ‘invasive’ as the patient is swallowing and introducing a chemical into their body which was either not present previously or in an incorrect quantity.

The other forms of treatment already known or being researched presently to treat mental illnesses are to use electric shock therapy and magnetic stimulation, both of which aim to work on the known electric activity of the brain, and to treat the illness through modifying or providing more electrical impulses into the brain, though it is not yet known how to treat each illness individually. Instead, the illnesses are blanketed by a single treatment with an electric shock to see whether it ‘works’ and helps the individual to return to a ‘normal’ state.

Mindfulness relates to being present and engaged in the moment, being aware of your thoughts and feelings without distraction or judgment. Mindfulness exercises have proved to be an effective therapy to treat many mental disorders, such depression and anxiety.

Identifying the regions in the brain activated during the exercises and modified with long term practice, could help in bringing an effective treatment. Targeting these regions by stimulating the neurons electrically or magnetically can provide a treatment for many mental disorders.

Electrical activity in the brain occurs through fluctuations resulting from ionic current within the neurons of the brain. The brain has such electrical properties during activities and when at rest. Understanding these properties can assist in influencing the brain to achieve one or more specific outcomes, for example, treating mental disorders or enhancing brain activity/performance.

It will be appreciated that one or more forms of the present invention advantageously provides a convenient arrangement for providing convenient, readily wearable non-invasive head covering for stimulating the brain of the wearer, which may be used to target one or more specific mental conditions of or to promote certain mental conditions, of the wearer.

It is against this background and the problems and difficulties associated therewith that the present invention has been developed.

### SUMMARY OF THE INVENTION

An aspect of the present invention provides a brain stimulator apparatus. The brain stimulator apparatus may have:
a power source;
a control system;
a plurality of electrodes;
a wearable support for the electrodes; and
a number of electrical conductors electrically connecting the electrodes to the control system and/or to the power source.

The control system may be configured to control application of electric charge to the electrodes via the electrical conductors, to produce electric current to induce stimulation of one or more neurons in the wearer's brain.

The control system may be configured to control application of electric charge to the electrodes via the electrical conductors, to produce electric current to induce stimulation of a cluster of neurons in the wearer's brain.

The electrodes can be distributed across the brain stimulator in a preferred arrangement to target one or more specific areas of the brain and/or one or more specific conditions or desired effects in the brain.

The electric charge applied can be a sequence of electric stimulation pulses. The sequence of electric stimulation pulses can be either voltage or current controlled pulses. The electric charge may also be applied as direct current stimulation.

The electrodes are preferably positioned evenly across the brain stimulator to target preferred areas of the brain, preferably to target left and right sides of the brain. The electrodes may be positioned similar or alike to those of an electroencephalogram (EEG).

The brain stimulator apparatus may be provided to stimulate the wearer's brain to promote a preferred mental condition, mood or emotion in the wearer. For example, electrical and/or magnetic stimulus may be applied to the wearer's brain to promote a positive emotion, such as a feeling of happiness or well-being (e.g. increase the release of certain neurotransmitters such as dopamine).

The brain stimulator apparatus preferably has electrically insulating material between the electrodes. Preferably the electrical insulating material isolates the electrodes from each other to prevent electrical interference.

The brain stimulator apparatus may include electromagnetic shielding to prevent or restrict external interference from one or more external electromagnetic fields (EMF).

The plurality of needles preferably insert into or contact onto a wearer's scalp for direct application of at least one electrical signal (e.g. voltage and/or current) to the wearer.

The brain stimulator apparatus may be provided to target specific neurons, neuron clusters and/or neuron pathways in the brain of the wearer.

The brain stimulator apparatus preferably includes a structure to conform to a wearer's head shape, for example, flexible fabric, mesh, stretchable fabric or other material, netting or a reticulated structure or material.

The brain stimulator apparatus is preferably attached to or attachable to headgear, such as a hat or cap. The headgear may be referred to as a wearable support. In some embodiments, the brain stimulator apparatus comprises the wearable support.

The power source preferably includes an electrical charge storage device, such as a battery and/or capacitive storage device.

The electrical charge storage device may be attached or attachable to the brain stimulator apparatus and/or to the headgear. The charge storage device may be replaceable and/or rechargeable.

The brain stimulator apparatus may include a processor for processing and controlling delivery of electrical signals to the brain and/or for receiving or monitoring electrical signals from the brain. The processor may be configured to control delivery of electrical signals to the brain and/or for receiving or monitoring electrical signals from the brain similar to or like an EEG.

The brain stimulator apparatus preferably includes wireless communication capability.

The brain stimulator apparatus may include wireless transmission and/or receiver capability, such as by short range data communication (e.g. Bluetooth, WiFi).

The brain stimulator apparatus may be arranged and configured to communicate with a mobile device, such as a mobile telephone, tablet device or laptop. The mobile device is preferably operated by the wearer. In some embodiments, the mobile device is in communication with the control system to control the voltage applied to the wearer via the electrodes. In some embodiments, the mobile device is in communication with the control system to monitor the voltage applied to the wearer via the electrodes. In some embodiments, the mobile device is in communication with the control system to control a current applied to the wearer via the electrodes. In some embodiments, the mobile device is in communication with the control system to monitor a current applied to the wearer via the electrodes. In some embodiments the mobile device is in communication with the control system to monitor cumulative charge applied to the wearer via the electrodes. The wearer may control the brain stimulator apparatus via the mobile device. For example, after the brain stimulator apparatus is calibrated, the user may be presented with a graphical user interface output on the mobile device that provides the wearer with one or more control options. The user may, for example, select to modify their brain activity, using the device, via the graphical user interface of the mobile device. That is, the graphical user interface of the mobile device may comprise a user-selectable button through which the user can control the brain stimulator apparatus.

In some embodiments, there is provided a method of stimulating the brain using a brain stimulator apparatus including a wearable brain stimulator, wherein: a plurality of electrodes contact onto or into a scalp of the wearer;
an assessment of a wearer's brain activity in at least a region of the brain is obtained via the electrodes;
and an electric charge is applied via the electrodes to cause electric current to stimulate the brain, parameters of the electric charge being determined based on the assessment.

In some embodiments, there is provided a method of stimulating the brain using a brain stimulator apparatus including a wearable brain stimulator, wherein: a plurality of electrodes contact onto or into a scalp of the wearer;
an assessment of a wearer's brain activity in at least a region of the brain is obtained via the electrodes;
and a current to stimulate the brain is applied through the electrodes, the current being determined from the assessment.

In some embodiments, both a current and a voltage are determined from the assessment. Both the voltage and the current may be applied through the electrodes.

The assessment may be a calibration assessment, such as to determine a baseline for applying the voltage and/or current. In other words, the assessment may comprise determining a baseline profile of the user's neural activity.

The assessment may be made via a mobile device (such as via an App running via a tablet, phone or other portable device) or via a clinical or other medical assessment. The assessment may be made by another computing device, such as a desktop computer.

A pattern for application of an electrical stimulus to the wearer's brain may identified, such as from the assessment or calibration. The pattern may include a combination of one or more electric pulse sequences and selection of regions for application of the one or more electric pulse sequences. The pattern may also include timing or order of application of the one or more electric pulse sequences.

The pattern may be programmed into the control system of the apparatus. The pattern may be determined using a machine learning model. The pattern may be determined using artificial intelligence. The pattern may be determined using another method and/or means.

The brain stimulator apparatus control system may be operated or influenced by the mobile device. In particular, the brain stimulator apparatus control system may be operated or influenced by software executed by a processor of the mobile device.

Preferably the controlled voltage and/or the current is applied according to the pattern, to the wearer's brain via the electrodes.

The control system may monitor and/or adjust the voltage and/or current, such as to avoid over stimulation.

The monitoring and/or adjustment of the controlled voltage and/or current may be controlled via the mobile device or controlled through the control system. The monitoring and/or adjustment of the voltage and/or current may be controlled via both the mobile device and the control system simultaneously.

### BRIEF DESCRIPTION OF THE DRAWINGS

One or more embodiments of the present invention will hereinafter be described with reference to the accompanying Figures, in which:

FIG. 1 shows an internal section view of the top of an embodiment of the invention.

FIG. 2 shows a detailed side view of an electrode of FIG. 1.

FIG. 3 shows a side view of an embodiment of FIG. 1 incorporating a specific feature.

FIG. 4 shows a flow chart of a method of an embodiment of the invention.

### DESCRIPTION OF PREFERRED EMBODIMENT(S)

It is to be appreciated that each of the embodiments is specifically described and that the present invention is not to be construed as being limited to any specific feature or element of any one of the embodiments. Neither is the present invention to be construed as being limited to any feature of a number of the embodiments or variations described in relation to the embodiments.

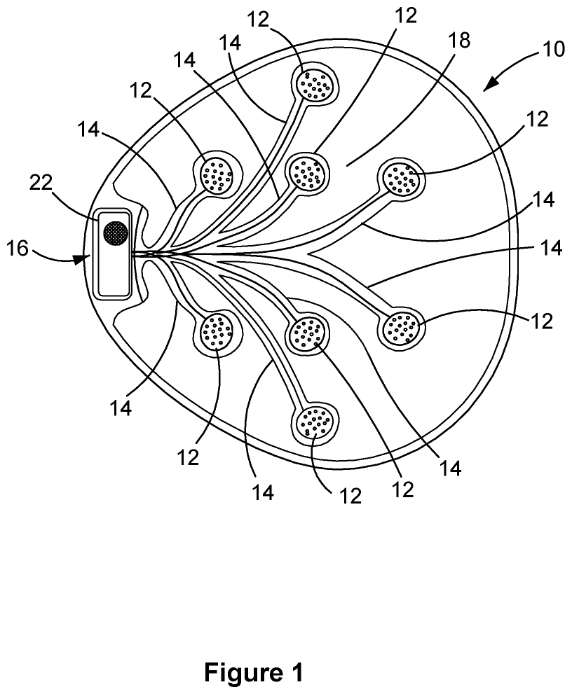

FIG. 1 shows a brain stimulator 10, according to some embodiments. The brain stimulator 10 may be referred to as a brain stimulator apparatus 10. In particular, FIG. 1 shows an internal structure of the brain stimulator 10. That is, a portion of an exterior (e.g. an outer skin) of the brain stimulator 10 is hidden in FIG. 1.

The brain stimulator 10 comprises a plurality of electrodes 12. The brain stimulator 10 is configured to generate one or more electrical outputs. The electrical output is an electric charge via the electrodes. The electrical output is configured to cause electric current in the wearer's head, thereby inducing stimulation of one or more neurons in the wearer's brain. In other words, the electrical output is configured to induce stimulation of a cluster of neurons in the wearer's brain. The electrical output comprises a voltage. The electrical output comprises a current. In some embodiments, the brain stimulator 10 is configured to apply the electrical output to one or more of the electrodes 12. In some embodiments, the brain stimulator 10 is configured to apply a respective electrical output to each electrode 12. For example, the brain stimulator 10 may be configured to generate a current and/or voltage and to apply the current and/or voltage to one or more of the electrodes 12, as part of a therapeutic treatment. In some embodiments, the brain stimulator 10 is configured to monitor the brain activity of the user via the electrodes 12, and generate an electrical output at a control system 16, the electrical output being indicative of the brain activity of the user. It will be understood herein that in either case, the electrical output may comprise one or more of a current and a voltage. In the case where the brain stimulator 10 applies the electrical output to the electrodes 12, the brain stimulator 10 may be said to be applying an electrical stimulus.

In the illustrated embodiment, the brain stimulator 10 that has eight electrodes 12. A first group of the electrodes 12 are configured to be electrically connected to a left side of a head of a user of the brain stimulator 10. The user may be referred to as a wearer. In the illustrated embodiment, four electrodes 12 are configured to be electrically connected to the left side of the user's head. These electrodes are configured to target areas of the left side of the user's brain.

A second group of electrodes 12 are configured to be electrically connected to a right side of the head of the user of the brain stimulator 10. In the illustrated embodiment, four electrodes 12 are configured to be electrically connected to the right side of the user's head. These electrodes are configured to target areas of the right side of the user's brain.

In some embodiments, the total number of electrodes 12 is four. The first group of electrodes 12 may comprise two electrodes and the second group of electrodes 12 may comprise two electrodes. In some embodiments, the total number of electrodes 12 is greater than four. For example, the total number of electrodes 12 may be any number between four and thirty six. In some embodiments, the first group comprises the same number of electrodes 12 as the second group. In some embodiments, the first group comprises a different number of electrodes 12 to the second group.

The brain stimulator 10 comprises a control system 16. The control system 16 comprises one or more processors. The control system 16 comprises memory. The one or more processors are configured to execute program instructions stored in the memory to cause the brain stimulator 10 to function as described herein. The one or more processor(s) may comprise one or more microprocessors, central processing units (CPUs), application specific instruction set processors (ASIPs), application specific integrated circuits (ASICs) or other processors capable of reading and executing instruction code.

The memory may comprise one or more volatile or non-volatile memory types. For example, memory may comprise one or more of random access memory (RAM), read-only memory (ROM), electrically erasable programmable read only memory (EEPROM) or flash memory. The memory is configured to store program instructions accessible by the one or more processor(s). The program instructions comprise executable program code modules. In other words, memory is configured to store executable code modules configured to be executable by the one or more processor(s). The executable code modules, when executed by the one or more processor(s) cause the brain stimulator 10 to perform certain functionality, as described in more detail below.

Each electrode 12 is electrically connected to the control system 16. In particular, each electrode 12 has a wire 14 connecting the electrode 12 to the control system 16. The control system 16 is configured to enable electrode data to be determined. In particular, the control system 16 may comprise hardware that enables the measurement of a voltage and/or a current at one or more of the electrodes 12. Such a voltage and/or current may be associated with brain activity of the user. This data may be stored in the memory. This data may be referred to as electrode data.

The placement of the electrodes 12 is specific to the type of condition being targeted. That is, if a person is suffering from depression, the electrodes 12 will be placed in a particular arrangement in the cap so that the desired areas of the brain that require electrical stimulation will be specifically targeted.

The brain stimulator 10 preferably includes a conformable material or structure such that the brain stimulator conforms to a shape of the wearer's scalp.

The brain stimulator can have an electrical isolation material 18 between the electrodes 12 to prevent interference between the electrical signals of each of the electrodes 12.

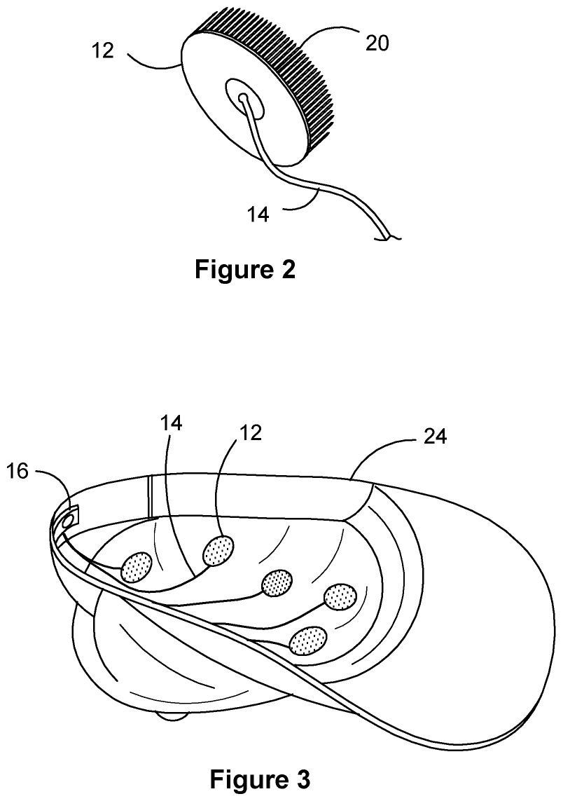

The electrodes 12 are shown in more detail in FIG. 2.

Preferably, one or more of the electrodes 12 has a number of small needles 20 protruding from the electrode 12. The needles are configured to insert into the skin of the head of the wearer, such as when the brain stimulator is tightly fitted to the wearer's head.

The needles 20 are present on the underside of the respective electrode 12, and the electrically insulated wires 14 provide the electrical stimulus to the wearer, and are preferably connected to the top side of the electrode 12.

In an embodiment, the electrical stimulus or voltage is provided to the wearer's brain via the needles 20 of the electrodes. The electrodes 12 will be positioned to target the particular health condition experienced by the wearer.

As described herein, the brain stimulator 10 is configured to apply an electrical output to each of the plurality of electrodes 12. In this way, the brain stimulator 10 may be said to deliver an electrical stimulation to the user when the brain stimulator 10 is electrically connected (e.g. via the electrodes) to the user's head. The electrical stimulation comprises a voltage. The electrical stimulation comprises a current. The electrical stimulation may be referred to as an electrical stimulus.

The electrical stimulus provided will be of a harmless/low voltage with desired current. It will preferably be selectable from a range of voltages. For example, a current intensity of less than 4 mA may be applied and varied during a period of use by the wearer. Further, the voltage of the electrical stimulus (i.e. the voltage applied by an electrode 12) may be varied between 1V and 4V, or across a range of values that are less than or equal to 9V. In some embodiments, the current of the electrical stimulus (i.e. the current applied by an electrode 12) may be varied between 1 mA and 4 mA. In some embodiments, the current of the electrical stimulus may be varied between 0 mA and 4 mA. One or both of the applied voltage and the current may be pulsed. One or both of the applied voltage and the applied current may be constant. One or both of the applied voltage and the applied current may be varied in another way. The voltage and/or the current may be varied by a voltage control unit. That is, the control system 16 may comprise a voltage control unit. An applied electrical signal can be pulsed or constant over a period of time. The applied electrical signal and/or pattern may be applied for one hour or less for stimulation purposes. The applied electrical signal and/or pattern may be applied for as long a time as is necessary for monitoring purposes.

The brain stimulator 10 comprises an electrical energy source. The electrical energy source may be referred to as a power source. The electrical energy source may be from an electrical charge storage device, such as a battery 22, which will be preferably relatively flat to maximise the level of comfort of the wearer and the wearability of the brain stimulator 10.

The battery 22 can be connected electrically to the control system 16, and either be connected in situ and able to be taken out and replaced, or it will be rechargeable.

The electrical signals are provided by the brain stimulator to excite the neurons of the brain in the specific targeted region so that the condition is mitigated, modified or controlled, at least to some extent.

The brain stimulator 10 may be worn at any time in which the patient is either being treated or feels the need to receive additional stimulus to ‘increase’ their mood from, for example, ‘depressed’ to ‘happy’.

The brain stimulator 10 can be inserted into a cap such as a baseball cap 24, in an embodiment, as shown in FIG. 3. This would facilitate the easy sliding on of the cap 24 to the patient's head and would enable the patient to wear the cap 24 whenever they so desired. The control system 16, will preferably have the capability to be connected wirelessly to a mobile application (an App) so that the patient is able to monitor and control the voltage and/or current (in-between threshold limitations set at the device) amount themselves, via their mobile device.

The brain stimulator 10 can be removably attached to the hat via a ‘hook and loop’ fastening system or clips or any other releasable fastening mechanism. In some embodiments, the brain stimulator is permanently attached to the hat.

Alternatively the brain stimulator 10 can be permanently attached to the hat via glue or sewn in, or any other manner of fastening permanently.

The brain stimulator 10 may have a mesh or any other material or adjustment having properties to allow stretching or expansion.

Preferably the diameter of the brain stimulator 10 is smaller than the diameter of the hat that the brain stimulator is removably or permanently attached to, allowing the wearer to easily place the hat on their head, whilst still maintaining a tight fit of the brain stimulator 10 to the wearer's skull.

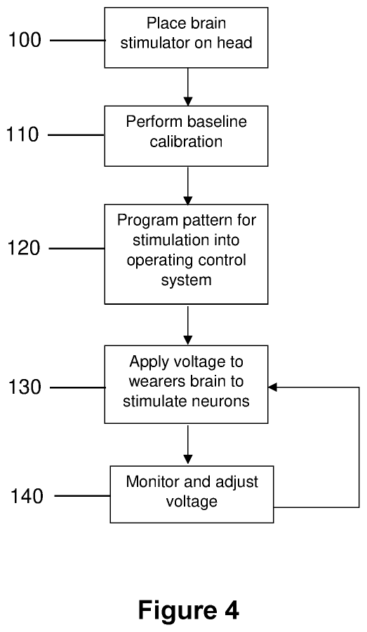

FIG. 4 illustrates via a flow chart, the method of stimulation application to a wearer of a brain stimulator.

Initially, the brain stimulator can be placed on the wearer's head 100, then the baseline calibration is performed 110. The one or more processors of the control system 16 may execute program instructions stored in the memory that are configured to cause the at least one processor to calibrate the brain stimulator 10. The one or more processors may calibrate the brain stimulator 10 based at least in part on electrode data determined via the plurality of electrodes 12. This calibration can involve mapping the sensed electrical activity of the brain in at least one state (i.e. resting, reading, walking, talking etc.) to provide baseline neural activity data. This baseline can be stored for comparison after treatment or to track changes in brain activity over time.

Analysis of this baseline can provide treatment requirement indications. For example, identifying regions with suppressed, overactive, or otherwise abnormal electromagnetic brain activity. Regions of abnormal activity may be targeted for stimulation treatment.

Alternatively, treatment may be based on known regions affected by disorders, such as depression or anxiety.

Once the baseline is obtained (i.e. once the brain stimulator 10 is calibrated), the pattern for stimulation indicating which areas of the wearers brain are to be stimulated is determined. This pattern may be referred to as an electrical stimulation pattern or sequence. The pattern may be referred to as a treatment pattern, a treatment electrical stimulation pattern and/or a treatment electrical stimulation sequence. The electrical stimulation pattern is associated with a time window over which the electrical stimulation pattern is to be applied. Parameters for the electrical stimulation can be based on target treatment. The electrical stimulation pattern comprises a respective electrical stimulation profile for each electrode 12. The electrical stimulation pattern comprises a voltage pattern. The voltage pattern comprises a voltage profile for each electrode 12. Each voltage profile is indicative of a voltage to be applied to a respective electrode 12 over the time window. The electrical stimulation pattern comprises a current profile for each electrode 12. Each current profile is indicative of a current to be applied to the respective electrode 12 over the time window.

The electrical stimulation pattern may be at least partially defined by one or more stimulation parameters. The stimulation parameters may take values indicative of one or more characteristics of the electrical stimulation pattern. For example, the stimulation parameters may comprise one or more of a number of pulses, an amplitude of one or more pulses, a duration of one or more pulses, a magnetic field intensity, which electrode 12 one or more of the pulses is to be applied to and a frequency of pulses.

In some embodiments, the pattern may be programmed 120 into the operating control system. Alternatively, a software algorithm is used to determine which area should be stimulated based on the baseline. In other words, a software algorithm (e.g. a machine learning model) may be used to determine the electrical stimulation pattern. In other words, Artificial Intelligence (AI) software may be used to determine which area should be stimulated based on the baseline and/or other data collected over a period of time. The other data may comprise data from one or more other patients with a known neurological condition, or previous data recorded with respect to the relevant wearer. The other data may be added to a brain activity profile of the wearer.

The wearer, the operator and/or the control system 16 then operates the voltage and/or current supply and applies voltage and/or current (i.e. the electrical stimulation) to the wearer's brain to stimulate 130 the desired areas/neurons in the brain. This may be done in accordance with a software recommendation. The voltage is monitored and adjusted 140 either automatically or via the mobile device (the App), to ensure the wearer is not over stimulated. That is, to ensure one or more of the electrical outputs of the brain stimulator 10 does not exceed a threshold value.

In other words, in some embodiments, the at least one processor of the control system 16 executes program instructions stored in the memory, the program instructions being configured to cause the at least one processor to apply an electrical output to each of the plurality of electrodes 12. The electrical output may correspond to the determined electrical stimulation pattern. For example, the electrical output applied to a particular electrode 12 may correspond to the voltage profile and/or the current profile determined as part of the electrical stimulation pattern.

There may also be the possibility of using magnets as the electrodes 12, to target the different areas of the brain using an electromagnetic field (EMF), so as to stimulate specific areas of the brain to treat the mental illness the patient is suffering from. In other words, each electrode 12 may comprise a magnet. In some embodiments, the electrode 12 may be in the form of a magnet. The magnet may comprise an electromagnet. That is, the magnet may comprise one or more electrically activatable coils. Current through the electromagnet may be applied so as to stimulate specific areas of the wearer's brain using an electromagnetic field. The electromagnetic field may be referred to as an electromagnetic stimulation principal field.

It is to be understood that, if any prior art is referred to herein, such reference does not constitute an admission that the prior art forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A brain stimulator apparatus having:
a power source;
a control system;
a plurality of electrodes;
a wearable support for the electrodes;
a number of electrical conductors electrically connecting the electrodes to the control system and/or to the power source;
wherein the control system is configured to:
calibrate the brain stimulator apparatus to determine a baseline profile of the user's neural activity, based at least in part on electrode data determined via the plurality of electrodes, wherein calibrating the brain stimulator apparatus comprises:
sensing electrical activity of a brain of the user, using the number of electrodes, over a sensing time window that is associated with at least one activity state; and
storing sensor data that is associated with the sensed electrical activity:
determine a treatment electrical stimulation pattern, based at least in part on the sensor data and other data that is associated with a known neurological condition, using a machine learning model, the treatment electrical simulation pattern comprising:
a current profile for each electrode, the current profile being indicative of a current to be applied to each electrode over a treatment time window; and/or
a voltage profile for each electrode, the voltage profile being indicative of a voltage to be applied to each electrode over the treatment time window; and
control application of a voltage to the electrodes in accordance with an electrical stimulation pattern, via the electrical conductors, in response to a treatment requirement indication, to, when worn, produce electric current through the wearer's head to induce stimulation of one or more neurons in the wearer's brain.

2. A brain stimulator apparatus according to claim 1, wherein the control system is configured to control application of a current to the electrodes via the electrical conductors, wherein the current applied stimulates one or more neurons in the wearer's brain.

3. A brain stimulator apparatus according to claim 1, wherein the electrodes are distributed across the brain stimulator to target one or more specific areas of the brain and/or one or more specific conditions or desired effects in the brain.

4. A brain stimulator apparatus according to claim 1, wherein the electrodes are positioned evenly across the brain stimulator to target preferred areas of the brain, targeting left and right sides of the brain.

5. A brain stimulator apparatus according to claim 1, wherein the brain stimulator apparatus has electrically insulating material between the electrodes, wherein the electrical insulating material isolates the electrodes from each other to prevent electrical interference.

6. A brain stimulator apparatus according to claim 1, wherein the apparatus includes electromagnetic shielding to prevent or restrict external interference from one or more external electromagnetic fields (EMF).

7. A brain stimulator apparatus according to claim 1, wherein one or more of the electrodes comprise a plurality of needles, the plurality of needles being configured to be inserted into or to contact a wearer's scalp for direct application of at least one electrical signal to the wearer.

8. (canceled)

9. A brain stimulator apparatus according to wherein the brain stimulator apparatus includes a structure to conform to a wearer's head shape, and the structure is made of a flexible fabric, reticulated structure or another solid material.

10. (canceled)

11. A brain stimulator apparatus according to claim 9, wherein the apparatus is attached to or attachable to headgear, wherein the each of the plurality of electrodes and the control system are configured to be connected to the headgear such that movement of the headgear causes corresponding movement of each of the plurality of electrodes and the control system.

12. (canceled)

13. A brain stimulator apparatus according to claim 11, wherein the power source includes an electrical charge storage device, a battery and/or capacitive storage device, wherein the electrical charge storage device is attached or attachable to the brain stimulator apparatus and/or to the headgear.

14. A brain stimulator apparatus according to claim 13, wherein the charge storage device is rechargeable.

15. A brain stimulator apparatus according to claim 1, wherein the apparatus includes a processor for processing and controlling delivery of electrical signals to the brain and/or for receiving or monitoring electrical signals from the brain.

16. A brain stimulator apparatus according to claim 15, wherein the apparatus includes wireless communication capability.

17. (canceled)

18. A brain stimulator apparatus according to claim 15, wherein the apparatus is arranged and configured to communicate with a mobile device, wherein the mobile device controls and monitors the voltage applied to the wearer via the electrodes.

19. (canceled)

20. (canceled)

21. (canceled)

22. A brain stimulator apparatus according to claim 1, wherein the voltage applied by the control system is pulsed or constant over a period of time.

23. A method of stimulating the brain using a brain stimulator apparatus including a wearable brain stimulator, the method comprising:
bringing a plurality of electrodes onto or into contact with a scalp of a wearer of the wearable brain stimulator;
conducting an assessment of the wearer's brain activity in at least a region of the brain using the electrodes, wherein conducting the assessment of the wearer's brain activity comprises determining electrode data that is associated with at least one activity state, via the plurality of electrodes;
calibrating the wearable brain stimulator to determine a baseline profile of the user's neural activity, based at least in part on the assessment;
determining a treatment electrical stimulation pattern, based at least in part on the electrode data and other data that is associated with a known neurological condition, using a machine learning model, the treatment electrical stimulation pattern comprising:
a current profile for each electrode, the current profile being indicative of a current to be applied to each electrode over a treatment time window; and/or
a voltage profile for each electrode, the voltage profile being indicative of a voltage to be applied to each electrode over the treatment time window; and
applying a voltage through the electrodes to stimulate the brain, the voltage being determined based at least in part on the treatment electrical stimulation pattern.

24. (canceled)

25. A method according to claim 23, wherein the treatment electrical stimulation pattern is programmed into the control system of the apparatus.

26. A method according to claim 23, wherein the brain stimulator apparatus control system is operated or influenced by a mobile device, wherein monitoring and/or adjustment of the voltage is controlled via the mobile device or controlled through the control system, and wherein the control system monitors and/or adjust the voltage, to avoid over stimulation.

27. (canceled)

28. (canceled)

29. (canceled)

30. A brain stimulator apparatus comprising:
a plurality of non-invasive electrodes that are configured to be electrically connected to a head of a user of the brain stimulator apparatus;
a wearable support configured to be connected to the electrodes;
a control system; and
a number of electrical conductors electrically connecting the electrodes to the control system,
the control system comprising:
at least one processor; and
memory storing program instructions accessible by the at least one processor and configured to cause the at least one processor to:
calibrate the brain stimulator apparatus to determine a baseline profile of the user's neural activity, based at least in part on electrode data determined via the plurality of electrodes, wherein calibrating the brain stimulator apparatus comprises:
sensing electrical activity of a brain of the user, using the plurality of electrodes, over a sensing time window that is associated with at least one activity state; and
storing sensor data that is associated with the sensed electrical activity in the memory:
determine a treatment electrical stimulation pattern, based at least in part on the sensor data and other data that is associated with a known neurological condition, using a machine learning model, the treatment electrical simulation pattern comprising:
a current profile for each electrode, the current profile being indicative of a current to be applied to each electrode over a treatment time window; and/or
a voltage profile for each electrode, the voltage profile being indicative of a voltage to be applied to each electrode over the treatment time window; and
apply an electrical output to each of the plurality of electrodes, in response to a treatment requirement indication, based at least in part on the treatment electrical stimulation pattern, over the treatment time window.

31. (canceled)

32. (canceled)

33. (canceled)

34. The brain stimulator apparatus of, wherein the at least one activity state is a resting state, a reading state, a walking state or a talking state.

35. (canceled)

36. (canceled)
